(19) United States
(12) Patent Application Publication
(10) Pub. No.: US 2026/0069842 A1
(43) Pub. Date: Mar. 12, 2026

(54) TISSUE EXPANDER SLEEVE AND TREATMENT FOR SHORT BOWEL SYNDROME, VAGINAL STENOSIS, VAGINAL AGENESIS / ATRESIA, AND NEOVAGINAL DILATION

(71) Applicant: Board of Supervisors of Louisiana State University and Agricultural and Mechanical College, Baton Rouge, LA (US)

(72) Inventors: Johnathan Steven ALEXANDER, Shreveport, LA (US); Giovanni Francesco SOLITRO, Shreveport, LA (US); Luke A. WHITE, Lafayette, LA (US); Stephanie Villalba, Stonewall, LA (US); Donald Sorrells, Shreveport, LA (US)

(73) Assignee: Board of Supervisors of Louisiana State University and Agricultural and Mechanical College, Baton Rouge, LA (US)

(21) Appl. No.: 17/901,847

(22) Filed: Sep. 1, 2022

Related U.S. Application Data
(60) Provisional application No. 63/217,437, filed on Jul. 1, 2021.

(51) Int. Cl. A61M 29/02

(52) U.S. Cl. CPC A61M 29/02

(57) ABSTRACT

A tissue expansion sleeve comprising a tube formed of woven polymer; the woven polymer forming helical trusses; and a biologically active material disposed on an outer surface of the tube.

### BACKGROUND

Short bowel syndrome (SBS) is considered the most common cause of intestinal failure in pediatric patients. Loss of small bowel length results in inadequate nutrient absorption due to a lack of functional surface area, leading to increased morbidity and mortality. The most common etiologies of short bowel syndrome in infant and pediatric patients include necrotizing enterocolitis, abdominal wall defects, jejunal ileal atresia, and mid gut volvulus. Bowel lengthening surgical procedures have been developed, however they carry substantial risks for morbidity due to their invasive nature and exclude patients with undesirable anatomy. Other methods of treatment include medications to slow intestinal transit and parenteral nutrition. Most patients are dependent on the parenteral nutrition route, which leads to risks such as sepsis, metabolic derangements and hepatic dysfunction. The amount of remaining bowel length has been shown to positively correlate with the ability to wean from parenteral nutrition. Despite the heavy and often mortal toll short bowel syndrome takes, there has yet to be a successful treatment shown in the current technology to heal the condition. For the foregoing reasons, there is a pressing, but seemingly irresolvable need for a treatment of short bowel syndrome.

### SUMMARY

Various objects, features, aspects, and advantages of the present invention will become more apparent from the following detailed description of preferred embodiments of the invention, along with the accompanying drawings in which like numerals represent like components. The present invention may address one or more of the problems and deficiencies of the current technology discussed above. However, it is contemplated that the invention may prove useful in addressing other problems and deficiencies in a number of technical areas. Therefore, the claimed invention should not necessarily be construed as limited to addressing any of the particular problems or deficiencies discussed herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fec. The accompanying drawings, which are incorporated in and constitute a part of the specification, illustrate various embodiments of the invention and together with the general description of the invention given above and the detailed description of the drawings given below, serve to explain the principles of the invention. It is to be appreciated that the accompanying drawings are not necessarily to scale since the emphasis is instead placed on illustrating the principles of the invention. The invention will now be described, by way of example, with reference to the accompanying drawings in which:

FIG. 1 shows a Roux-en-Y surgical model using the disclosed intestinal expansion sleeve (IES) to promote gut elongation. The same type of tissue extender would be placed in the blind limb, except that in Aim 2, tissue extenders would contain GLP2 to enhance gut restitution. Arrows show chyle flow outside of the blind limb.

FIGS. 2A-2D are four microphotographs and FIG. 2E is four charts showing histology of the small and large intestine with IES resulted in significant wall thinning in all layers of the small and large intestines in response to dilation of the lumens with IES. FIG. 2A shows small intestine undilated. FIG. 2B shows small intestine dilated. FIG. 2C shows large intestine undilated. FIG. 2D shows large intestines dilated. FIG. 2E shows graphical numeric representation of the width of the various tissues of the small and large intestines for both dilated and undiluted. Bar=300 μm. Comparisons made by t-test between non-expanded and expanded states for total thickness and each tissue layer.

FIGS. 3A-3C are three photographs showing mechanical characterization of the DE device during the expansion phase at strains of 50% (FIG. 3A), 25% (FIG. 3B), and at its fully relaxed/biased nominal length (FIG. 3C).

FIGS. 4A and 4B are two photographs showing representative sleeve and introduction into intestine (FIG. 4A), and intestine after expansion of the sleeve (FIG. 4B).

FIG. 5 is a photograph showing mechanical testing of an IES bowel connection, where the IES at top (blue) is sutured to intestinal wall (lower section) and load failure performance is analyzed.

FIG. 6 is three graphs that show sleeve placement in rabbit small intestine versus length of IES (n=3 for each group).

FIG. 7 is two graphs that show sleeve placement in rabbit colon versus length of IES (n=3 for each group).

FIGS. 8A-8I are nine photographs showing one embodiment of attaching the IES to small intestines to treat short bowel syndrome.

FIG. 9 is a perspective view of a tissue expansion sleeve embodying features of the present invention.

FIG. 10A is a front cross-sectional view of the embodiment of the tissue expansion sleeve depicted in FIG. 9.

FIG. 10B is a perspective cross-sectional view of the embodiment of the tissue expansion sleeve depicted in FIG. 9.

FIG. 10C is another perspective cross-sectional view of the embodiment of the tissue expansion sleeve depicted in FIG. 9 in a stretched state.

FIG. 11A is a side-view photograph of a first prototype of a tissue expansion sleeve embodying features of the present invention.

FIG. 11B is a top-view photograph of the prototype of a tissue expansion sleeve depicted in FIG. 11A.

FIG. 11C is a top perspective-view photograph of the prototype of a tissue expansion sleeve depicted in FIG. 11A.

FIG. 12A is a side-view photograph of a second prototype of a tissue expansion sleeve embodying features of the present invention.

FIG. 12B is a top perspective-view photograph of a cap for use with the prototype of a tissue expansion sleeve depicted in FIG. 12A.

FIG. 12C is a side perspective-view photograph of the prototype of a tissue expansion sleeve depicted in FIG. 12A with the cap installed.

FIG. 13A is an ex vivo photograph of an animal test subject showing post-insertion of a tissue expansion sleeve into the test subject.

FIG. 13B is an ex vivo photograph of an animal test subject showing post-insertion of a tissue expansion sleeve into the test subject and sutured into place.

FIG. 13C is an ex vivo photograph of an animal test subject showing the harvested vaginal canal of the test subject with the tissue expansion sleeve still inserted.

FIG. 14A is a graph showing the comparative thickness of the vaginal wall layers of an animal test subject with and without a prototype of a tissue expansion sleeve made in accordance with the teachings of the present invention.

FIG. 14B is a microphotograph of the thickness of the vaginal wall layers of an animal test subject without a prototype of a tissue expansion sleeve made in accordance with the teachings of the present invention.

FIG. 14C is a microphotograph of the thickness of the vaginal wall layers of an animal test subject with a prototype of a tissue expansion sleeve made in accordance with the teachings of the present invention.

FIG. 15 is a perspective view of a single layer, highly porous tissue expansion sleeve embodying features of the present invention.

### DETAILED DESCRIPTION

The present invention will be understood by reference to the following detailed description, which should be read in conjunction with the appended drawings. It is to be appreciated that the following detailed description of various embodiments is by way of example only and is not meant to limit, in any way, the scope of the present invention. In the summary above, in the following detailed description, in the claims below, and in the accompanying drawings, reference is made to particular features (including method steps) of the present invention. It is to be understood that the disclosure of the invention in this specification includes all possible combinations of such particular features, not just those explicitly described. For example, where a particular feature is disclosed in the context of a particular aspect or embodiment of the invention or a particular claim, that feature can also be used, to the extent possible, in combination with and/or in the context of other particular aspects and embodiments of the invention, and in the invention generally. The terms “comprise(s),” “include(s),” “having.” “has,” “can,” “contain(s),” and grammatical equivalents and variants thereof, as used herein, are intended to be open-ended transitional phrases, terms, or words that do not preclude the possibility of additional acts or structures. are used herein to mean that other components, ingredients, steps, etc. are optionally present. For example, an article “comprising” (or “which comprises”) components A, B, and C can consist of (i.e., contain only) components A, B, and C, or can contain not only components A, B, and C but also one or more other components. The singular forms “a,” “and” and “the” include plural references unless the context clearly dictates otherwise. Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

The term “at least” followed by a number is used herein to denote the start of a range beginning with that number (which may be a range having an upper limit or no upper limit, depending on the variable being defined). For example, “at least 1” means 1 or more than 1. The term “at most” followed by a number is used herein to denote the end of a range ending with that number (which may be a range having 1 or 0 as its lower limit, or a range having no lower limit, depending upon the variable being defined). For example, “at most 4” means 4 or less than 4, and “at most 40% means 40% or less than 40%. When, in this specification, a range is given as “(a first number) to (a second number)” or “(a first number)-(a second number),” this means a range whose lower limit is the first number and whose upper limit is the second number. For example, 25 to 100 mm means a range whose lower limit is 25 mm, and whose upper limit is 100 mm.

The embodiments set forth the below represent the necessary information to enable those skilled in the art to practice the invention and illustrate the best mode of practicing the invention. For the measurements listed, embodiments including measurements plus or minus the measurement times 5%, 10%, 20%, 50% and 75% are also contemplated. For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The term “substantially” means that the property is within 80% of its desired value. In other embodiments, “substantially” means that the property is within 90% of its desired value. In other embodiments, “substantially” means that the property is within 95% of its desired value. In other embodiments, “substantially” means that the property is within 99% of its desired value. For example, the term “substantially complete” means that a process is at least 80% complete, for example. In other embodiments, the term “substantially complete” means that a process is at least 90% complete, for example. In other embodiments, the term “substantially complete” means that a process is at least 95% complete, for example. In other embodiments, the term “substantially complete” means that a process is at least 99% complete, for example.

The term “substantially” includes a value is within about 10% of the indicated value. In certain embodiments, the value is within about 5% of the indicated value. In certain embodiments, the value is within about 2.5% of the indicated value. In certain embodiments, the value is within about 1% of the indicated value. In certain embodiments, the value is within about 0.5% of the indicated value.

The term “about” includes when value is within about 10% of the indicated value. In certain embodiments, the value is within about 5% of the indicated value. In certain embodiments, the value is within about 2.5% of the indicated value. In certain embodiments, the value is within about 1% of the indicated value. In certain embodiments, the value is within about 0.5% of the indicated value.

In addition, the invention does not require that all the advantageous features and all the advantages of any of the embodiments need to be incorporated into every embodiment of the invention.

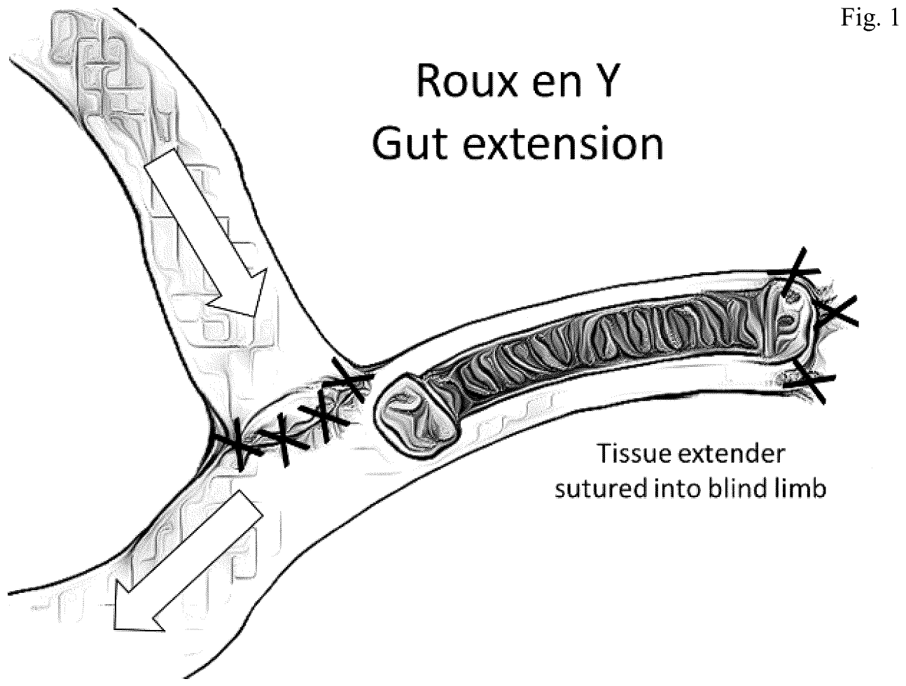

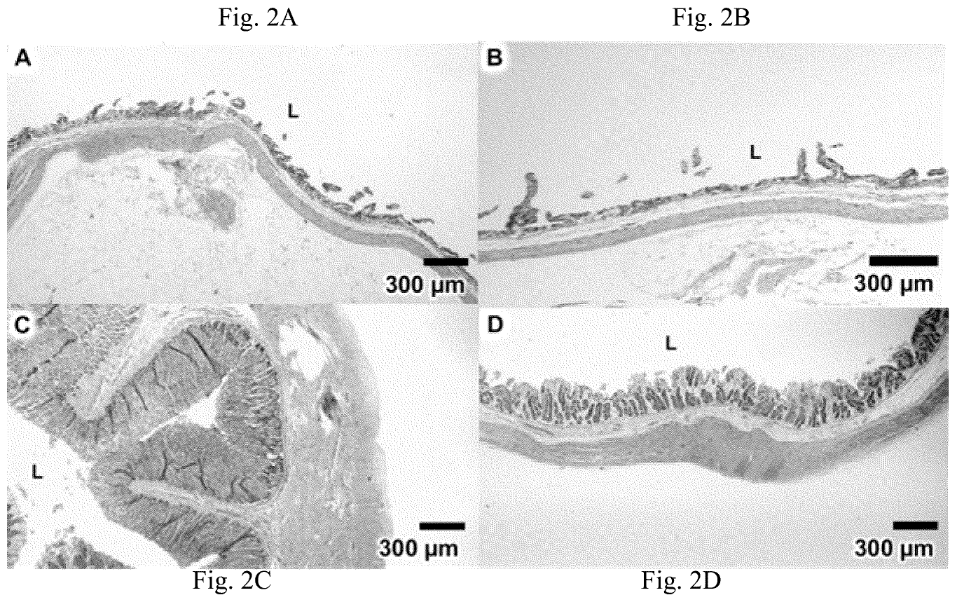

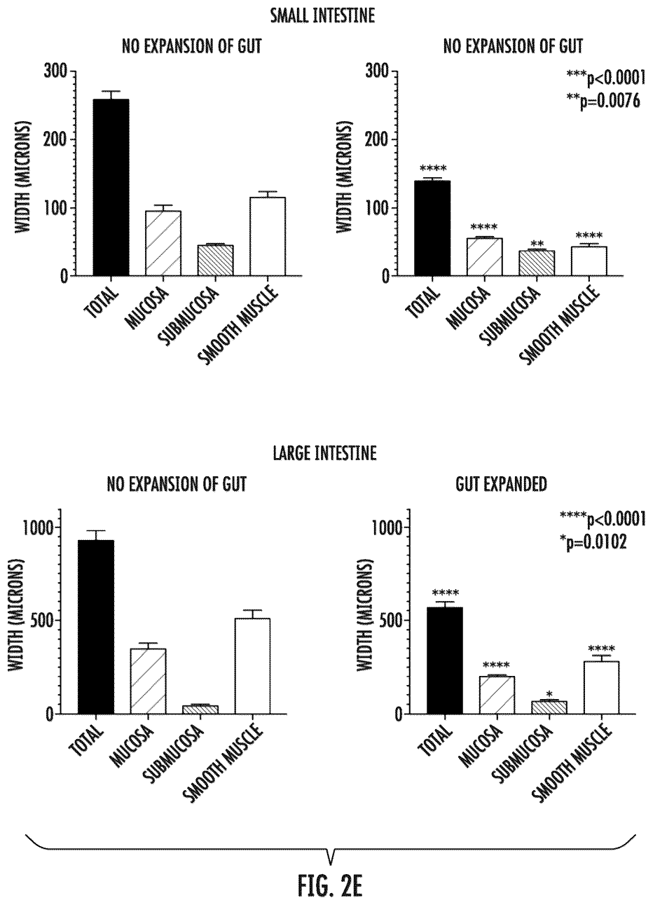

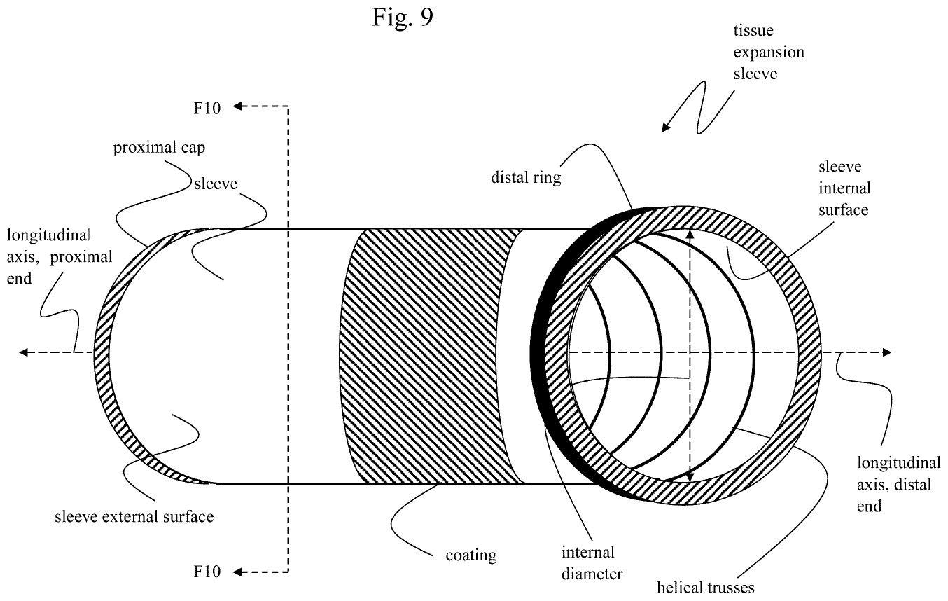

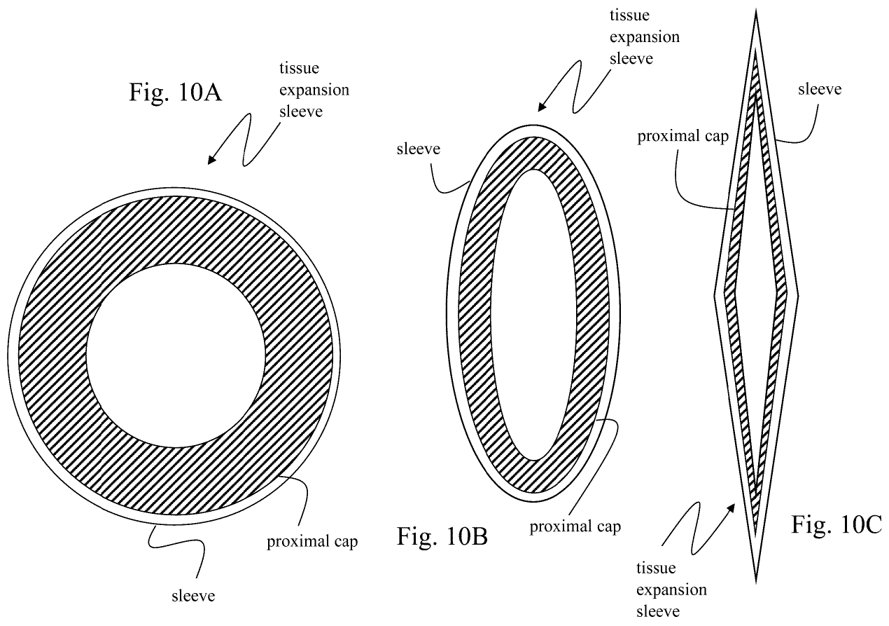

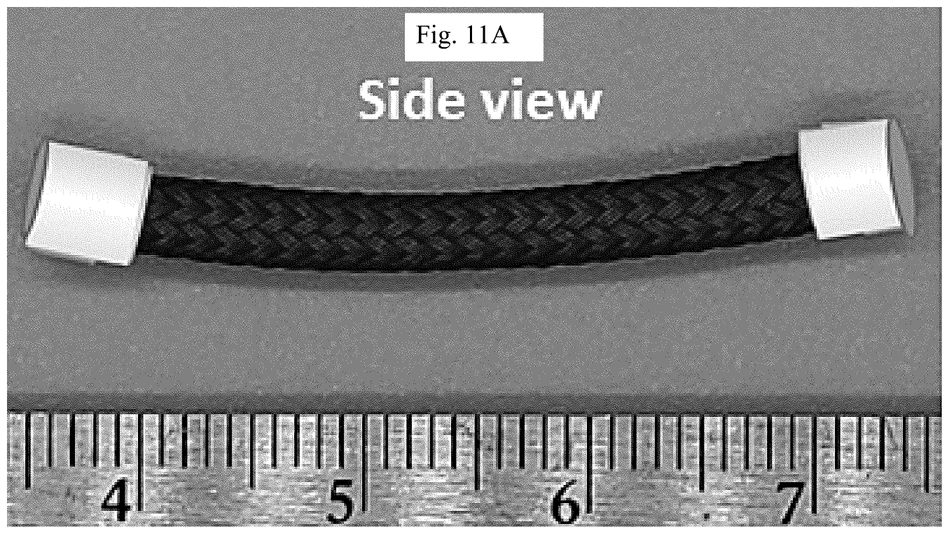

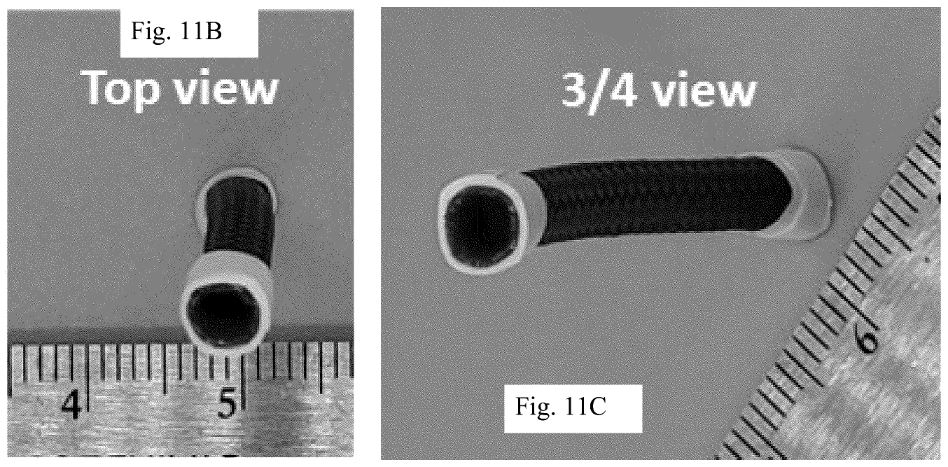

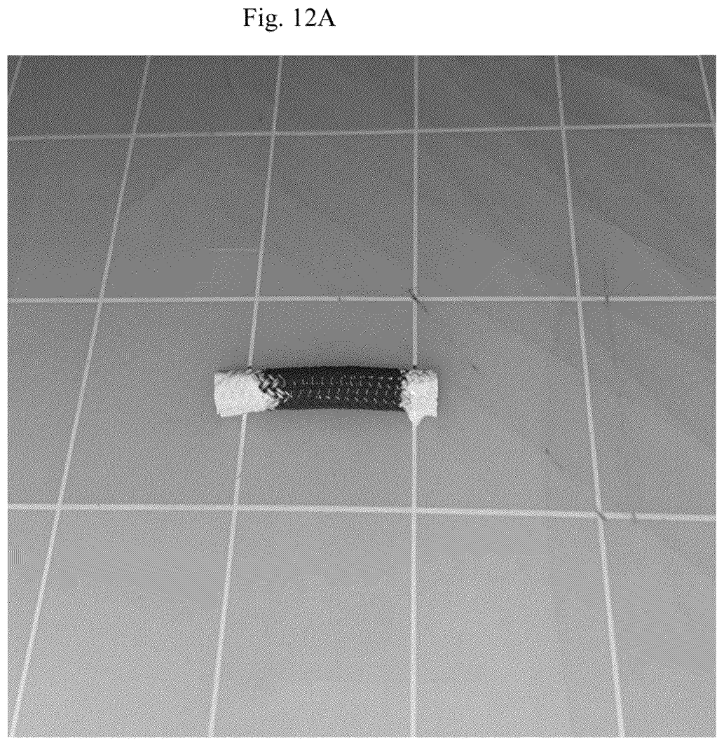

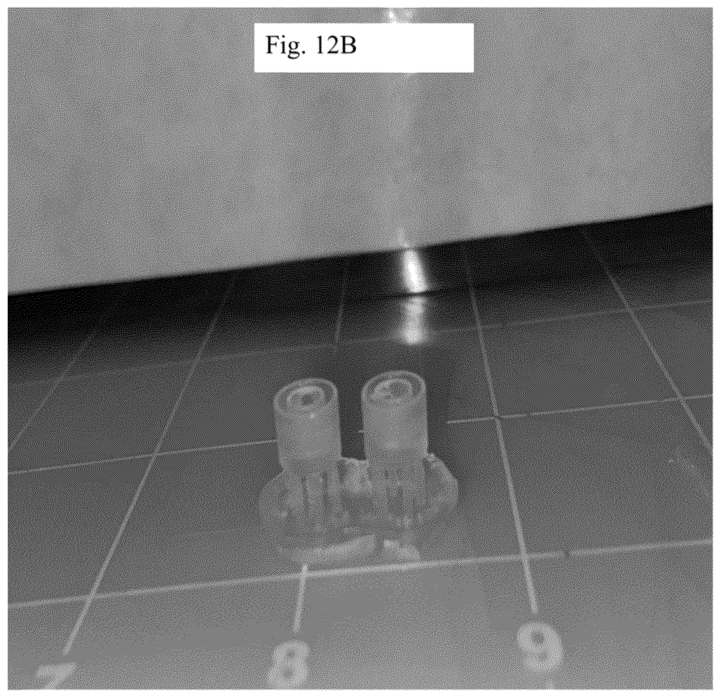

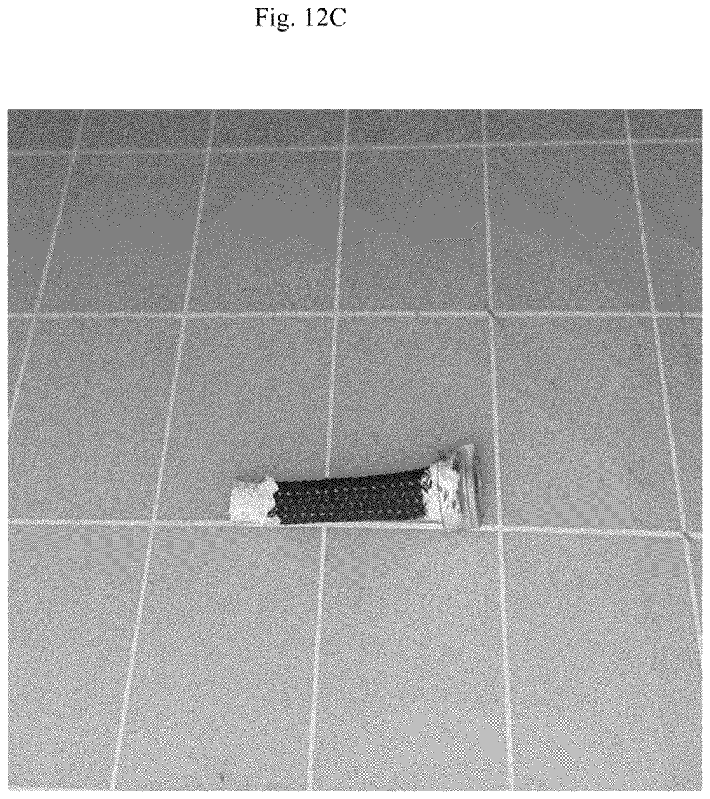

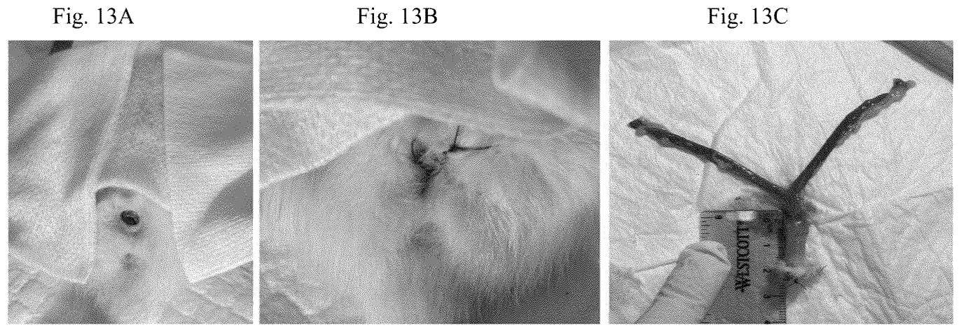

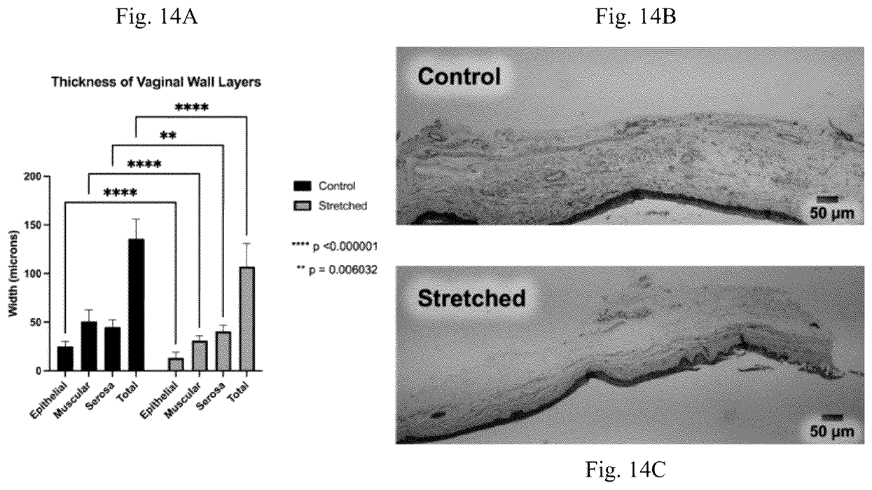

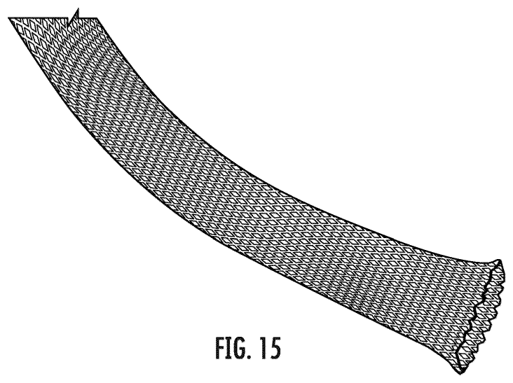

Turning now to FIGS. 1-11C, a brief description concerning the various components of the presently disclosed inventions will now be briefly discussed.

Many disease processes (e.g., necrotizing enterocolitis, caustic esophageal injury, malrotation with volvulus) can result in short-gut syndrome (SGS) where remnant intestinal segments may dilate radially, but rarely elongate longitudinally. Here the inventors mechanically characterize a novel model of a self-expanding mesh intestinal expanding sleeve (IES) for use in SGS.

Gut lengthening was achieved using a novel cylindrical layered woven polyethylene terephthalate intestinal expansion sleeve (IES) device with helicoid trusses with isometric ends. The IES is pre-contracted by diametric expansion, deployed into the gut and anchored with bioabsorbable sutures. The IES then expands slowly against radial tension from the tissue until the IES reaches its equilibrium dimension and maintains longitudinal gut tension. The slow, gentle, and persistent manner of the lengthening permits tissue remodeling, increases absorptive surface area, and preserves vascular and nervous supplies. The inventors performed mechanical testing to obtain the effective force-displacement characterization achieved on these prototypes and evaluated minimum numbers of sutures needed for anchoring in some embodiments. Furthermore, the inventors deployed these devices in small and large intestines of New Zealand White rabbits, measured IES length-tension relationships, and measured post-implant gut expansion ex vivo. Histology of the gut before and after implantation was also evaluated. In further studies, the IES was used in experiments to successfully, gently lengthen vaginal canals in sheep.

Longitudinal tension using IES did not result in suture failure. Maximum IES suture mechanical loading was tested using 4-6 sutures; the inventors found similar failure loads of 2.95±0.64, 4±1.9 and 3.16±0.24 Newtons for 4, 6 and 8 sutures respectively (n=3, n.s.). Pre-contracted IES tubes were deployed at 67±4% of initial length (i.1.); in the large bowel these expanded significantly to 81.5±3.7% of i.1. (p=0.014, n=4). In the small bowel, pre-contracted IES were 61±3.8% of i.1.; these expanded significantly to 82.7±7.4% of i.1. (p=0.0009, n=6). This resulted in an immediate 24±7.8% and 36.2±11% increase in gut length when deployed in large and small bowels respectively with maintained longitudinal tension. After the initial immediate increase in gut length, more permanent tissue remodeling occurs. Maintained IES induced tension produced gut wall thinning in the short term.

The IES is a versatile platform for gaining length in SGS, which may be simply deployed via feeding tubes. Our results need further validation for biocompatibility and mechanical characterization to optimize use in gut expansion.

Distraction enterogenesis (DE) is a method whereby small intestine elongation is accomplished by the use of longitudinal mechanical force to provide a therapeutic option for those with SBS. A major challenge with DE has been developing a method which minimizes radial expansion while maximizing longitudinal lengthening. Previous methods of attempting to cause longitudinal bowel lengthening have been limited by their transmission of radial force against the bowel wall, which leads to risks such as perforation and necrosis secondary to compression of the blood supply of the bowel wall. The excessive radial force component of these previous methods has led to significant challenges in translating basic science experiments to clinically applicable technology.

Despite its challenges, DE has promise in obtaining sustained intestinal lengthening with concomitant functionality. This preserved functionality is characterized by the presence of mesenteric neovascularization, muscular hypertrophy, increased epithelial cell proliferation, and increased villus height and crypt depth. Early studies testing DE in pigs required creation of blind-ending segments of intestine or the use of vessels loops or full thickness sutures to facilitate mechanical force transmission to the bowel wall. These methods potentially required multiple separate open operations to both place and remove the devices or restore intestinal continuity, which adds substantial surgical complexity and potentially results in the loss of gained intestinal length after anastomosis creation.

Pediatric surgeons often encounter patients with intestinal failure due to inadequate intestinal length. These patients are challenging because the adaptive process for reaching enteral goals is slow. Patients with short gut compensate with gastroparesis and slow dilation of the intestinal diameter. This process may take weeks, months or years requiring supplemental parenteral nutrition for adequate growth. Native elongation of the short intestine usually is limited. Two surgical elongation procedures have been developed, but both procedures require that the patient has developed sufficient dilation of bowel diameter. The medical care of these patients is encumbered by daily needs for total parenteral nutrition (TPN), central line access, and metabolic derangements.

One possible solution to managing this clinical dilemma is to initiate an intestinal elongation strategy early. DE enables elongation of the intestine in animal models. Initial studies have these devices placed out of continuity of the gastrointestinal tract such as in a Roux limb. The potential improvement in the clinical course of these patients is tremendous with decreased days on TPN, decreased need for central access and decreased line infections.

A method and device invented by the inventors to address some or all of the drawbacks in the prior art is a tissue expansion sleeve (TES). The TES is a linear tissue expander. The sleeve is a unique expansion device for tubular structures. In one embodiment, the TES is an intestinal expansion sleeve (IES). In other embodiments the TES is a vaginal expansion sleeve (VES). With regards to short gut syndrome, there are several potential benefits/advantages of the IES over other strategies. The IES is a tube with a porous wall, making successful placement in continuity likely. The contracted IES expands radially in proportion to the amount of shortening in length of the IES. The radial expansion makes the IES easier to secure to the intestine in a noninvasive embodiment. Lastly, the fully expanded sleeve has a decreased diameter making eventual passage of the IES in the stool more possible.

The inventors' IES DE device is an implantable porous mesh walled sleeve, which, according to one embodiment, is attached at each end of the device to the intestine with absorbable sutures. The IES is designed to exert a linear force curve as it expands, thereby lengthening the intestine. The IES device segment is designed to eventually be passed in stool once the absorbable sutures have dissolved and the IES has extended. According to one embodiment, to save the patient operations and potential intestinal length, the device is attached or otherwise held to the end of a tube and deployed in the intestines without surgery. The tube may be, for example, a transpyloric feeding or a colonoscope, each of sufficient length to reach the desired location from the access opening. Multiple deployments of these sleeves are possible. In this disclosure, the applicants evaluate the achieved force-strain relationship in the applicants' prototypes, evaluate the achievable tensioning force in relation to the number of sutures used for its anchoring, and present the results of a study placing the novel sleeve in rabbit intestine. The inventors predict based on the experimental results that an expansion greater than 40% of the initial implanted IES device length is achievable with no more than six sutures.

This disclosure describes an apparatus and a method for deployment of a woven nylon, plastic or metal mesh tube which has the property that when expanded diametrically the length of the tube is reduced proportionately. This allows the tube to be placed onto a deployment sheath which holds the tube in the ‘open’ or compressed conformation, which is, for example, 20%-40% shorter, more preferably 30% shorter than when in a biased expanded conformation. The tube may then be inserted into the gut and the ends of the IES may be sutured into the gut wall. The stent ends are then compressed together slightly which allows the coil to release from the stent and the sheath is removed. When the stent is ‘relaxed’ it attempts to lengthen against the gut wall and produces significant longitudinal stretch which immediately lengthens the gut by ˜20%. This is not the full lengthening capacity however and if allowed to fully expand, the gut segment will be lengthened by up to 50%, for example. After gut remodeling, this would achieve an improvement of gut length which is the significant problem in short bowel syndrome.

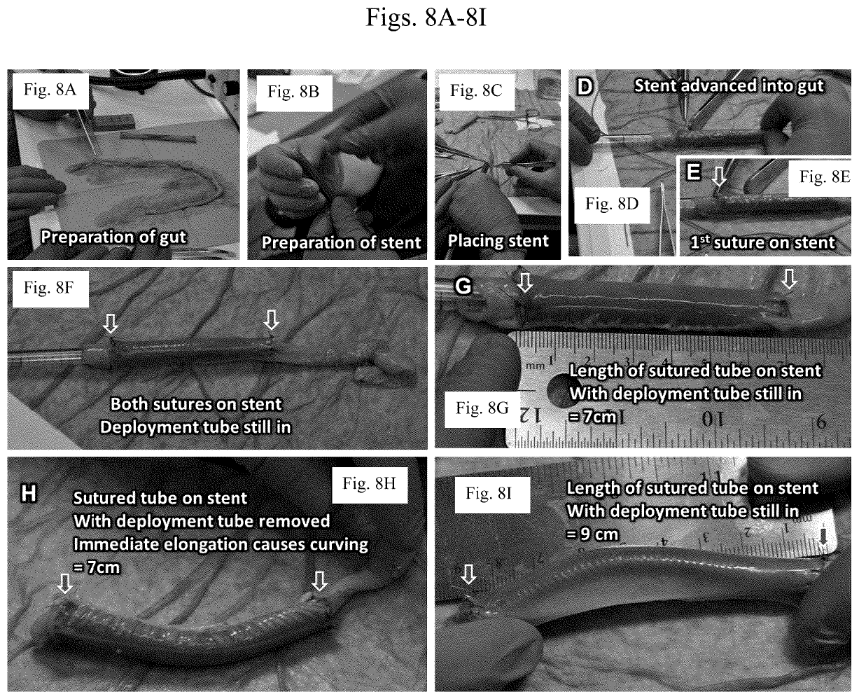

FIGS. 8A-8I show the introduction of a pre shortened segment of an IES mesh tube, which decreases the tube length, but increases its diameter. In this longitudinally compressed, or shortened but widened state, the IES tube is introduced into a segment of gut (FIG. 8C). Following its deployment into the segment of the gut which is to be extended, the shortened tube on its guide is then sutured into place (FIGS. 8D and 8E) in the gut while the gut is under normal tension. Following suturing at both ends (FIG. 8F), the stent is, in this embodiment, 7 cm in length (FIG. 8G). After the deployment guide is removed (FIG. 8H) the tube can increase in length immediately to, for example, 20-25% longer than the initial compressed loading length (FIG. 8I, 9 cm in this embodiment). This produces significant lengthening tension in the gut. This leads to a persistent lengthening by longitudinal tension in the gut which over time results in an extension of the gut. The gut is anticipated to fully remodel during the placement of this device, achieving full clinical benefit.

Short gut syndrome (SGS) is a clinical phenomenon affecting about 30,000 individuals each year where gut length is significantly reduced, compromising the ability to sustain adequate gastrointestinal nutrient absorption. This syndrome usually reflects the catastrophic loss of intestinal tissue from a volvulus (twisting and ischemia of the intestine), infection (necrotizing enterocolitis), immune disorders (Crohn's disease), trauma and idiopathic causes. The small intestine absorbs most nutrients and adapts to tissue loss by dilating in diameter which increases surface area for absorption but not length. Children and adults require years to become independent of IV nutrition. Sometimes SGS-afflicted individuals never become fully independent of IV nutrition/total parenteral nutrition (TPN). The cost for IV nutrition and/or hospitalization to treat line infections is substantial. In the long term. SGS patients may develop multiple central line infections, liver disease and organ failure.

The Medicare cost per short gut patient receiving TPN varies between $64,000 to $128,000 annually. For the 30,000 US patients receiving TPN, this amounts to societal cost of $1.9 billion (about $6 per person in the US) annually. This figure only includes hyperalimentation solution and does not consider costs for hospitalizations, labs, and surgeries. These patients are frequently impaired from fully contributing to society through a vocation of their choice. This cost to society is difficult to measure but present.

The disclosed intestinal expansion sleeves, a type of tissue expansion sleeve along with, for example, vaginal expansion sleeves, have tremendous potential to increase the length of residual intestine in such patients. With longer intestinal length, these patients gain independence from TPN, require less hospitalizations and decrease need for central venous lines with significant improvement in quality-of-life scores. Distraction enterogenesis has the potential to treat short bowel syndrome.

Our IES offers multiple improvements compared to prior devices. The sleeve is porous and is deployable in continuity with GI tract. Also, the IES will not require removal in some embodiments, as it will be held in place with absorbable sutures. The device has limited radial expansion and excellent linear expansion as shown by this study. Lastly, the IES enables the device to be deployed without surgery over a feeding catheter as well as coated with drugs to promote healing and/or decrease inflammation.

### Competition and Advantage

Currently there are no IES devices in testing on humans. Distraction enterogenesis is a new field of study and the inventors have published a unique methodology for achieving IES distraction (gut lengthening, Sorrells et al., 2021). Only one group, Dunn et al. has employed metal springs to achieve bowel lengthening in any way even tangentially related to the inventors' device. There are several critical advantages to the inventors' prototype over the typical spring. First, IES sleeve diameters diminish as gut lengthening is achieved. This allows for spontaneous and easy passage and elimination of the device through the GI tract after elongation. Second the sleeve material can be coated with materials which can enhance the process of gut lengthening. Further, the IES sleeves may be constructed out of materials that dissolve over time in the body, further aiding in passage after a set amount of time. IES sleeves have tremendous surface area to bind drugs compared to metal springs. Third, as the device is contracted, the diameter enlarges slightly. This is a potential property of the sleeve which may enable a future nonsurgical method of deployment of the sleeve into the intestine.

### Ease/Cost of Proof-of-Concept

The inventors' lab has demonstrated distraction using IES sleeves in rabbit intestinal tissue, among other experiments. Using our approach, we can gain significant increases in gut length up to 30%. Mechanical testing performed in our lab also determined the optimal number of sutures to secure the device. The inventors' lab has recently continued to evaluate this model with testing in an ex vivo rat model. These hollow, tubular IES sleeves are 30 mm in length and compress to approximately 20 mm. Therefore, our IES sleeve has the potential to gain 50% increase in gut length over time. This approach allowed us to implant expansion sleeves in a Roux limb of intestine on the rat in vivo. The Roux-en-Y procedure isolates the sleeve in a limb of intestine which is unlikely to result in iatrogenic complications such as peritonitis from erosion, bowel obstruction, or fistulae. Once the IES sleeve creates length while maintaining gut structure, we place sleeves in continuity. These experiments were repeated using IES which are infused with GLP-2, a hormone which is used to promote gut healing and absorptive capacity.

Vaginal Lengthening with Tissue Expansion Sleeve

Occasionally, a female infant is born with a defect in the development of the vagina. This presents a significant challenge for the surgeon correcting the maldevelopment. One major class of this anomaly is known as a cloacal defect. Essentially the rectum, bladder and vagina come together to form a single channel. The surgery in infancy to repair this defect is difficult, delicate, and lengthy. Often these infants have a shortened vaginal with the repair. Or the infant may have a piece of intestine brought down to serve as the conduit. This vagina canal does not grow with the child and necessitates either a dilation process or further surgery to make the conduit more amenable to intercourse. These solutions are limited on their success.

Recently, our lab has developed a tissue intestinal expansion sleeve that allows for expansion of intestinal segments. The sleeve when placed in rabbit intestine shows a 35% increase in initial length ex vivo. Current attempts at elongating the vagina have resulted in more dilation than elongation. This is obviously a functionally inferior result. The inventors disclose herein the tissue expansion sleeve functioning as a vaginal expansion sleeve does function to lengthen the vaginal canal in mammals. The VES is expected to revolutionize the lengthening the vaginal canal in humans.

Some infants are born with a shortened or nonexistent vaginal canal. The VES can lengthen the vaginal canal allowing natural intercourse, etc. The VES may also be used to address the conditions of vaginal stenosis, vaginal agenesis/atresia, and function as a neovaginal dilation. These conditions especially benefit from the large surface area of the mesh, allowing for extensive hormone or other therapeutic loading on the VES sleeve.

The benefit is immediate for any patient needing vaginal reconstruction, for any patient having had vaginal reconstruction surgery and requiring dilation, and for patents that experience vaginal stenosis, such as with menopause. The potential to aide all of these patients is hard to quantify on an emotional wellness scale.

### Methods

### Sleeve Preparation and Characterization:

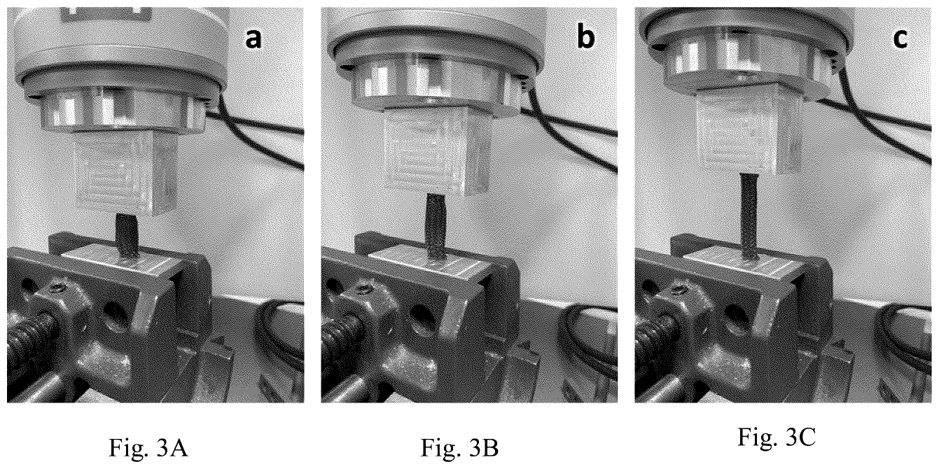

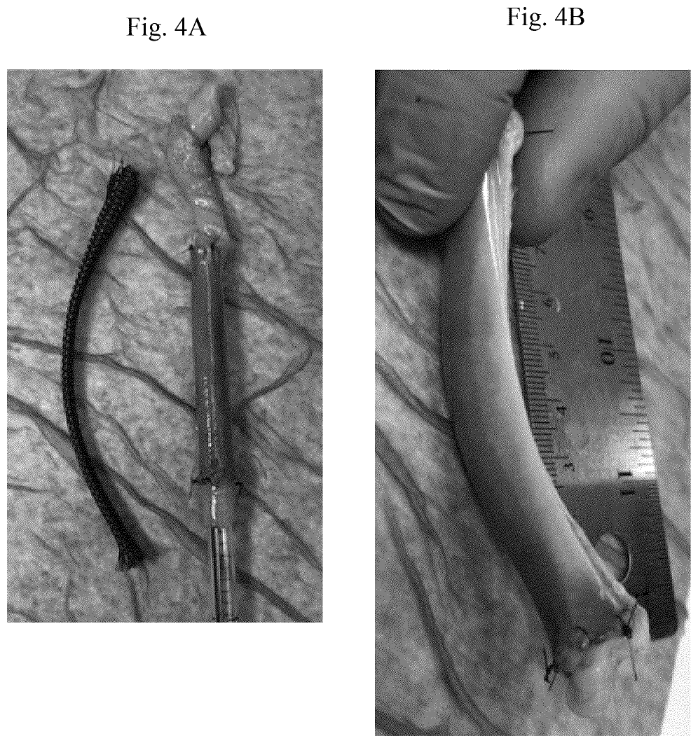

Intestinal expansion sleeves (IES) were produced as cylindrical layered polyethylene tubes with helical trusses. The ends of the sleeves were heat-treated to smooth the edges. These sleeves are shown in FIGS. 4A and 4B. Edges of sleeves were inspected and milled smooth as needed. The sleeve lengths were measured individually in their native form (initial length, ‘i.1.’). Mechanical characterization was performed using an Instron 8874 Biaxial Servo Hydraulic Fatigue Testing System, applying a cycle of compression followed by expansion at a rate of 50 mm/min (see FIGS. 3A-3C). With reference to the nominal length of the DE devices, force values were recorded during the expansion phase at strain values 10, 20, 30, 40 and 50%.

### Implantation of the Sleeves:

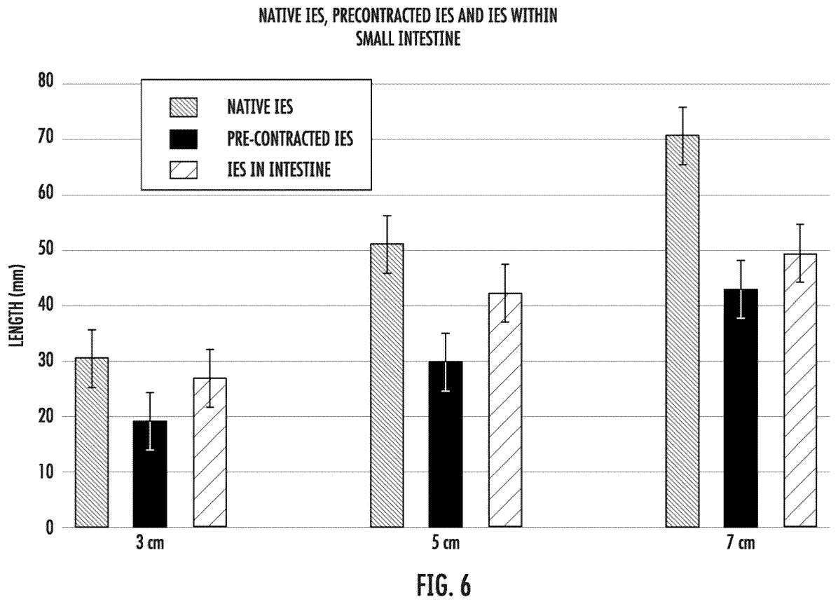

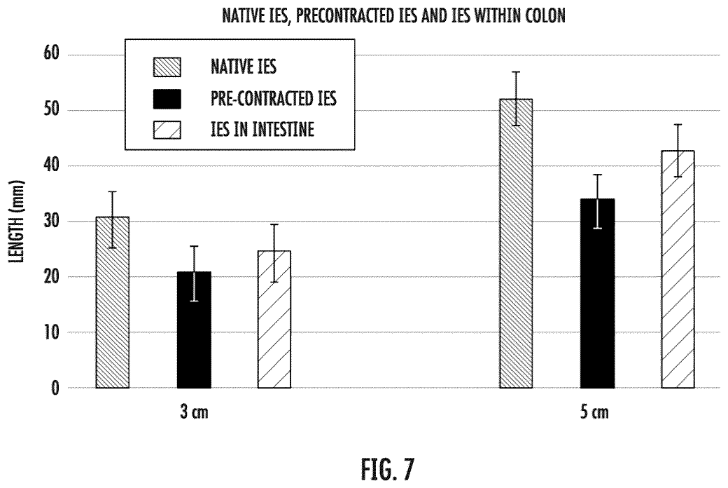

Using small and large intestines from New Zealand White rabbits, several test sleeves were implanted. Intestines were harvested from recently sacrificed rabbits and lumen washed with isotonic saline. Measurements of the intestine in native form were obtained, and sleeves then placed over a 0.8 cm plastic tube with an end tapered to 3 mm (FIGS. 4A and 4B). The placement of the sleeve over the tubing resulted in shortening of the sleeve to its pre-contracted form which was recorded. An enterotomy was made in intestinal segment and the tube containing the pre-contracted sleeve was placed into the intestinal segment. Four sutures (4-0 polydioxanone, Ethicon) were placed at both ends of the IES to secure the sleeve inside the intestinal wall in the pre-contracted state. Removal of the pipette resulted in immediate partial expansion of the sleeve within the intestinal segment; the extent of bowel length extension was measured using calipers. These data are shown in FIGS. 6 and 7.

### Mechanical Testing of the Bowel/Device Connection:

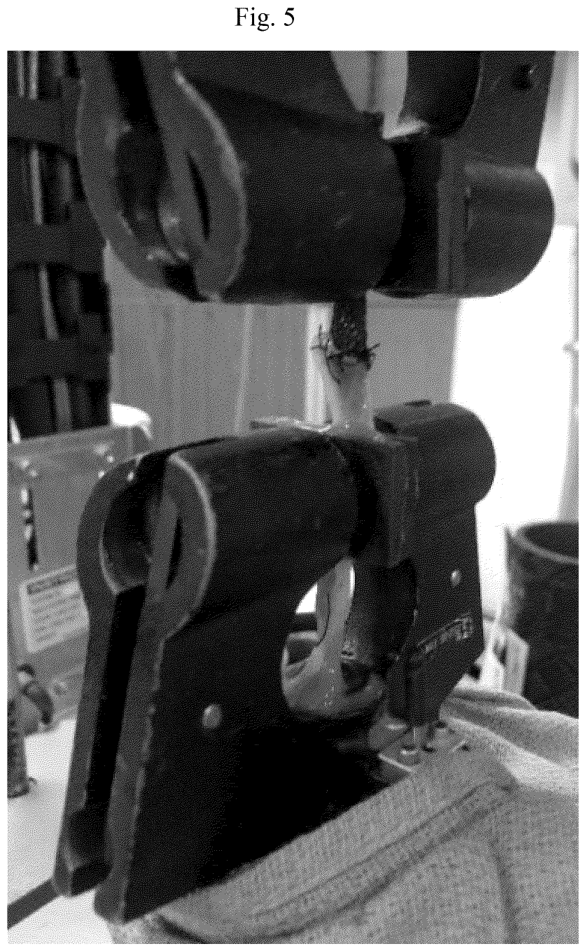

Intestinal segments instrumented with the IES were tested to determine maximum force prior to failure in relation to the number of sutures used for the anchoring of the IES. The testing was performed on the same Instron machine used for the characterization. The specimens, consisting of an IES connected to a bowel segment were held in place by two pneumatic grips at 25 psi of pressure as shown in FIG. 5. FIG. 6 is a graph of sleeve placement in the rabbit small intestine versus length of IES (n=3 for each group). Testing to failure was performed by delivering tension to the specimens at a rate of 50 mm/min until failure. The three configurations tested included IES devices connected with 4, 6 or 8 sutures. Load displacement data were recorded at a frequency of 100 Hz and for every 0.1N increments. The peak load measured during testing was considered as the failure load for the specimen.

### Results

### Mechanical Characterization of the Created Prototypes:

The tested prototypes exerted forces that varied from 0.73±0.28N for the 10% strain to a peak of 6.44±1.05N for the 50% strain (p=0.01, see Table 1). The 30 and 40% strains, respectively with loads of 2.31±0.35N and 2.84±0.36N were significantly different (p=0.01) but comparable in amplitude to the failure loads later measured for the bowel.

TABLE 1
Load values [N] recorded at various strain values
for each tested nominal length of the DE device.

Strain

| DE nominal length | 10% | 20% | 30% | 40% | 50% |
|---|---|---|---|---|---|
| 30 | 0.46 | 1.61 | 2.32 | 2.82 | 6.08 |
| 30 | 0.62 | 2.07 | 2.84 | 3.31 | 7.29 |
| 30 | 0.85 | 2.37 | 2.99 | 3.54 | 7.59 |
| 40 | 0.10 | 1.10 | 1.82 | 2.26 | 3.99 |
| 40 | 0.70 | 1.76 | 2.14 | 2.83 | 7.63 |
| 40 | 1.01 | 1.83 | 2.18 | 2.78 | 6.57 |
| 50 | 0.98 | 1.91 | 2.23 | 2.78 | 6.41 |
| 50 | 0.94 | 1.77 | 2.15 | 2.55 | 5.78 |
| 50 | 0.91 | 1.80 | 2.10 | 2.67 | 6.64 |
| Average | 0.73 | 1.80 | 2.31 | 2.84 | 6.44 |
| SD | 0.28 | 0.32 | 0.35 | 0.36 | 1.06 |

The bowel alone showed a failure load of 2.48±0.17N that was not significantly different from the failure load of 2.95±0.52N found for the IES/bowel connection performed with 4 sutures (p=0.10). Compared to the connection with 4 sutures, the increment to 8 resulted in a failure load of 3.16±0.20N that was found not significantly different (p=0.31, see Table 2). The failure of the system under tension revealed that failure was mainly localized in the bowels and was independent of suture numbers.

TABLE 2
Bowel/device connection

| | | Localization of the Failure |
|---|---|---|
| Number of Sutures | Failure Load [N] | Formation |
| intact bowel | 2.75 | Proximity of the grip |
| intact bowel | 2.31 | Mid-bowel |
| intact bowel | 2.38 | Mid-bowel |
| Intact bowel | 2.49 | Mid-bowel |
| 4 | 2.85 | bowel |
| 4 | 3.64 | bowel |
| 4 | 2.37 | stitches |
| 6 | 2.46 | bowel |
| 6 | 3.33 | bowel |
| 6 | 6.25 | stitches |
| 8 | 3.42 | stitches |
| 8 | 2.94 | bowel |
| 8 | 3.12 | bowel |

### Animal Model:

Six (6) sleeves were placed in colons and 9 sleeves were placed in small bowels. Measurements were taken by a caliper and lengths recorded in mm. The sleeves used in the colon showed a pre-deployment decrease in length of 67±3.6% of i.1. The sleeves expanded in the colon to 81.6±3% of i.1. of the sleeve (p=0.014, n=6). In the small bowel pre-contracted IES were 60.6±3.7% of i.1.; these expanded significantly to 80.2±8.4% of (p=0.0009, n=9). This resulted in an immediate 24±7.8% and 36.2±11% increase in gut length of the colon and small bowel respectively.

Average increase of intestinal length for 3 cm and 5 cm sleeves in the small intestine was 39±3% and 44±13% respectively. Interestingly, the percent increase in length for 7 cm was only 16±8%. The colon data were similar (FIG. 7, n=3 in each group). The IES colon percent increase in i.1. was 19±8.5% for 3 cm sleeve and 26.7±3.5% for 5 cm.

We accomplished load testing on IES sutures in small bowel with 4, 6 or 8 sutures. Failure under load as defined by a sudden drop in tension was seen at 2.95±0.64, 4±1.9 and 3.16±0.24 Newtons for each respective number of sutures. There was no significant difference between these groups regarding failure load. There was no apparent relationship between the number of sutures and the failure load indicating that using four sutures to secure the IES device in the intestine may be safe and may not be improved by additional suturing. The failure load of the small intestine alone was 4±1.5 Newtons.

The expansion force was measured for sleeve lengths of 3, 5, and 7 cm for the small intestine. We found that the expansion force at 10% differed significantly from 20%, 30%, 40% and 50% at a length of 30 mm. Similarly, at 50 mm, the expansion force again differed significantly between 10% and other values 20%, 30%, 40% and 50% values of compression. At the intermediate length of 40 mm, 10% compression was only significantly different from the 50% compression.

### Discussion

Mechanical data (Table 2) show that average failure load for native rabbit small intestine was 2.48±0.19N. Failure load with four fixation sutures was similar and ⅔ of failures occurred in bowel and not the connection of the tensiometer to intestine (n=9). Increasing the number of sutures to the IES device did not significantly affect the failure load. These data support four fixation sutures for the IES device.

Our strain data on prototypes of varying lengths show that the sleeves exceed the failure load of rabbit intestine when compressed more than 30% (Table 1). Some widening of helical trusses and dampening of the load values of IES are suggested prior to placing in vivo in New Zealand white rabbits.

Our data demonstrate a significant immediate expansion of the intestine (small bowel or colon) and the resilience of the suture fixation of the IES. Mechanical testing also demonstrated the load properties of the intestine before elongation by the IES. This information is important to the design of IES devices. Preferably, the force (N) of expansion of the IES should be less than the failure load of the intestine. Histology shows that stretching of the intestinal wall reduces wall thickness and flattening of individual wall layers, which may influence perfusion and wall tension; these factors need to be more fully evaluated in additional studies. Designing IES devices with sub intestine failure load force dynamics will decrease the potential for intestinal perforation.

When different lengths of IES are compared, the initial expansion benefit appears to dimmish at the 7 cm (16±8%) vs 5 cm (44±13%) length. This seems to indicate an optimal length for the IES and that longer lengths are not always better. The key to gaining maximum length in tissue expansion in some embodiments is deployment of multiple IES devices or serial deployments over time.

### Vaginal Expansion Sleeve (VES)

Successful creation of neovagina without the trauma of invasive surgery and without the patient vigilance of mechanical dilation.
VES device: Adopted mechanical characteristics of IES device; Woven cylindrical layered sleeve with helicoid trusses; Coated ends with flex seal-barium mixture
Biomechanical Characterization: Instron 8874 Biaxial Servo Hydraulic Fatigue Testing System. Tested force load of VES at 50%, 40%, 30%, 20%, 10% compression.
Biomechanical Results: Mean nominal length: 30.1 mm±0.3. Mean re-expansion length: 29.9 mm±0.3. Mean nominal length: 30.1 mm±0.3. Mean re-expansion length: 29.9 mm±0.3

| Compression | 50% | 40% | 30% | 20% | 10% |
|---|---|---|---|---|---|
| Force (N) | 11.7 ± 3.5 | 2.0 ± 0.1 | 1.2 ± 0.1 | 0.7 ± 0.1 | 0.3 ± 0.1 |

Ex Vivo Insertion: Sprague-Dawley rats. VES pre-contracted, inserted into vaginal canal, secured using 3 non-absorbable 4-0 silk sutures, allowed to re-expand. Vaginal canals harvested post-implant and new canal lengths measured.

Ex Vivo Vaginal Canal Expansion: Mean pre-insertion length: 18.3 mm±3.4; Mean post-insertion length: 23.6 mm±2.4. Mean vaginal lengthening of 5.4 mm±1.2, a 32.5±23.6% increase.

| | 1 | 2 | 3 | 4 | 5 | 6 | 7 |
|---|---|---|---|---|---|---|---|
| Pre-insertion (mm) | 13 | 16 | 16 | 22 | 21 | 21 | 19 |
| Post-insertion (mm) | 21.5 | 22 | 26 | 25 | 25 | 26 | 20 |
| Expansion (%) | 65.4 | 37.5 | 62.5 | 13.6 | 19.0 | 23.8 | 5.3 |

Immediate vaginal elongation was successfully achieved with VES. VES expansion to its equilibrium dimension maintained longitudinal vaginal canal tension, which permits tissue remodeling and increased canal length while preserving vascular and nervous supplies.

### Conclusions

The TES is therapeutic for multiple embodiments, for example in surgical applications for increasing the length of small or large bowel, which has been shortened by, for example, necrotic injury followed by surgical excision leading to a shorter gut segment which causes deficiencies in absorption and nutrition. Other embodiments include using the IES device for use in creating and/or elongating the vaginal canal for individuals who have vaginal agenesis or shortened vaginal canals. This would be treatment for individuals with Mayer-Rokitansky-Kuster-Hauser (MRKH) syndrome or complete or partial androgen insensitivity syndrome (AIS), for example. Further, the VES may be used for lengthening surgically created neovaginas for MRKH or AIS patients, or for transgender female patients. The VES may be employed directly after surgery, with preferably a sequential use of multiple widths and or lengths as the vaginal canal is stretched longer and/or wider. Further, for individuals who have experienced vaginal atrophy/atresia, due to decreased estrogen post menopause for example, the VES can gently stretch the vagina to regain depth and width. The long-term gentle stretching of the VES is contrasted starkly with the repeated short term and forceful dilation that is standard care for gaining or maintaining vaginal length, often with pain and other physical and psychological complications.

The VES may be coated with GLP2, steroids, one or more compounds that increase epithelial or endothelial growth, such as growth factors, drugs, steroids, and such. The VES may further be coated with estrogen to promote tissue elasticity and flexibility, increased vaginal secretions, restoring lubrication, and decrease vaginal pH, to normal post puberty and pre-menopause levels for post-menopausal women, or to promote vaginal tissue genesis with neovaginas.

The VES may be inserted into the vagina such that a proximal end of the VES extends to the proximal end of the vagina (toward the interior or the cervix for individuals who have a cervix). The distal end of the VES preferably is secured adjacent to but inside of the vaginal introitus. In one embodiment the distal end of the VES has a larger diameter and/or transverse cross-section than a majority of the remainder of the VES, to aid the VES in maintaining secure position inside the vaginal canal, while the VES gently and continuously stretches the vagina inward. The distal end of the VES may also have a textured and/or higher friction coefficient external surface, with preferably a wider cross section, to aid in securing the VES in location inside the vaginal introitus. Further, the wider cross section provides a greater area with which the distal end of the VES may press on the interior wall of the vagina adjacent to the vaginal introitus, reducing the pressure on the tissue at the location while providing sufficient anchoring force to push inward.

Preferably the distal end is firm but elastic, such that it may be folded to insert into the inside of the vaginal canal, and then automatically unfold to a biased shape, such as a circle or oval, preferably larger in diameter than a vaginal introitus opening, and firm enough to substantially maintain shape while exerting force against the vaginal wall adjacent the vaginal introitus. In a further embodiment, the proximal end of the VES may be shaped to comfortably press against the proximal end of the vaginal canal. In a further minimal internal gap embodiment, the VES may have an elliptical or oval or rhomboid cross section, to minimize open interior space in the vagina while being stretched, while also fully, gently stretching the vaginal wall. In a further minimal internal gap embodiment, a first interior wall of the VES remains substantially in contact with a second opposite interior wall of the VES as the VES expands inside the vagina, with the VES contracting transverse to a VES longitudinal axis (or radially) as the VES expands along the longitudinal axis. In further embodiments, the exterior cross-sectional edges of the VES are rounded, even in rhomboid cross sections, so as to not irritate the vaginal wall.

In further embodiments, alternative or additional chemicals, cells, and or therapeutics may be coated on the surface of the VES, such as progesterone, LH, FSH, estrogen, wound healing chemicals, antibiotics, buffers and/or other chemicals to maintain a pH in the vagina of below 7, more preferably below 6, more preferably below 5 and more preferably below 4.5, and most preferably between 4.5 and 3.8. Stem cells may also be disposed on the surface of the VES for transfer to the vaginal walls. Such cells includes circumcision skin cells, other stem cells, including mesenchymal stem cells, including adipose derived mesenchymal stem cells and placental mesenchymal stem cells including human placental amnion derived mesenchymal stem cells and human placental villous derived mesenchymal stem cells, including human placental multipotent mesenchymal stromal cells, and biological signals that guide secretory products, including immunoregulatory cytokines, growth factors and exosomes into the desired tissue, such as vaginal mucosa epithelial tissue, for example.

The VES may be changed out every week, two weeks, four weeks, one month, two months, three to twelve months, and 1.00 to 5.00 years. In a further embodiment, a protein scaffold may be woven onto or otherwise attached to the exterior of the sleeve to aid in transfer of MSCs loaded on the scaffold to the tissue that the tissue elongation sleeve is in contact with, for example, vaginal epithelial cells. In a further embodiment, repeated treatment with VES of different sizes, with protein scaffold loaded with MSCs thereon. This is an approach that will improve the results for surgical neovaginas. In further embodiments, a specialized panty may be worn with a reinforced crotch area, to prevent exit of the VES.

### Structural Description:

The TES is preferably a woven polymer mesh and has helicoid trusses and elastic properties. Is not a spring, but similar. When the sleeve is longitudinally contracted, the diameter increases, by 1-4 mm, for example, or up to 1.5 its original size. A benefit is that you can pick very calibrated specific dilators for specific sized tissue tubes. If a sleeve is contracted by 40%, it may take a 4 mm dilator inserted into the center longitudinal axis inside the TES, applying outward radial pressure on the TES, causing the TES to expand outward radially and contract axially in an exact and very reproducible manner. In such a scenario, a 20% contraction would be made with smaller dilator, and a 50% contraction with larger diameter dilator inserted inside and through the interior of the TES. An issue with a spring is you can't put anything in the middle to cause it to contract. But this mesh will reliably contract radially based on how you stretch the diameter of sleeve with a dilator. With most human tissues, there is a goldilocks amount of force. Too much force and you damage tissue. To little force and no physiological response happens. This sleeve provides a reliable, rapid manner of determining how much force you want to give a tissue based on how much you stretch the center outward, and how much potential energy is stored in the sleeve. Different diameters of deployment yield different contraction and thus different expansion when inserted.

Because the sleeve is porous and has a much higher surface area than a spring and a much greater amount of drug or therapeutic may be loaded thereupon. The sleeve may be constructed wholly or partially out of a woven biodegradable material-such as PDS, and preferably sealed on both axial ends. Then the TES could be inserted, and, depending on the thickness of the weave and the type of material, the TES may last 3-5 months in place, for example, and then degrade on its own.

A schematic diagram of one embodiment of the tissue expansion sleeve 100 is shown in FIGS. 8A-I.

The invention illustratively disclosed herein suitably may explicitly be practiced in the absence of any element which is not specifically disclosed herein. While various embodiments of the present invention have been described in detail, it is apparent that various modifications and alterations of those embodiments will occur to and be readily apparent those skilled in the art. However, it is to be expressly understood that such modifications and alterations are within the scope and spirit of the present invention, as set forth in the appended claims. Further, the invention(s) described herein is capable of other embodiments and of being practiced or of being carried out in various other related ways. The present disclosure also contemplates other embodiments “comprising.” “consisting of and “consisting essentially of,” the embodiments or elements presented herein, whether explicitly set forth or not. In addition, it is to be understood that the phraseology and terminology used herein is for the purpose of description and should not be regarded as limiting. The use of “including.” “comprising,” or “having” and variations thereof herein is meant to encompass the items listed thereafter and equivalents thereof as well as additional items, while only the terms “consisting of and “consisting only of are to be construed in the limitative sense.

Wherefore, I/we claim:

1. A tissue expansion sleeve comprising
a tube formed of woven polymer;
the woven polymer forming helical trusses; and
a biologically active material disposed on an outer surface of the tube.
